(19) United States
(12) Patent Application Publication
(10) Pub. No.: US 2025/0213443 A1
(43) Pub. Date: Jul. 3, 2025

(54) CLEANSING AGENT

(71) Applicant: SHISEIDO COMPANY, LTD., Tokyo (JP)

(72) Inventors: Shinichiro NAKANO, Tokyo (JP); Rie KURINOBU, Tokyo (JP)

(73) Assignee: SHISEIDO COMPANY, LTD., Tokyo (JP)

(21) Appl. No.: 18/687,396

(22) PCT Filed: Sep. 15, 2022
(86) PCT No.: PCT/JP2022/034523

§ 371 Date: Mar. 10, 2025

(30) Foreign Application Priority Data
Sep. 28, 2021 (JP) 2021-158289

(51) Int. Cl. A61K 8/36; A61K 8/72; A61K 8/92; A61Q 19/10

(52) U.S. Cl. CPC A61K 8/361; A61K 8/72; A61K 8/92; A61Q 19/10; A61K 2800/596

(57) ABSTRACT

The present invention provides a cleansing agent that can generate foam having excellent texture and can give an excellent sensation during use after cleansing. The cleansing agent includes (A) an anionic surfactant, (B) a wax having a melting point of 60° C. to 93° C., and (C) a cationic polymer.

### TECHNICAL FIELD

The present invention relates to a cleansing agent suitable for skin cleansing such as face cleansing. More specifically, the present invention relates to a cleansing agent that can generate foam having excellent texture and can give an excellent sensation during use.

### BACKGROUND ART

A surfactant such as a higher fatty acid soap is generally incorporated in a cleansing agent used for face cleansing or the like from the viewpoint of cleansing power and foaming properties. In particular, in a facial cleansing agent, in order to improve a sensation during use, moisture retention, and the like in addition to cleansing power, additional combination with a fragrance, a viscosity modifier, a moisturizing agent, and other additives has been studied.

In a cleaning agent, bubble characteristics largely depend on a type and amount of a surfactant, but sufficient properties in terms of texture of foam, sensation during use after cleansing, and the like may not be obtained with only a surfactant. That is, ease of foaming, elasticity of foam, ease of rinsing after cleansing, smooth feeling of the skin after cleansing, and the like may be insufficient. In order to improve such points, it has been studied to select a type of a surfactant or to combine with a water-soluble polymer (for example, Patent Literature 1 to 3). However, in particular, in cleansing agents intended for face cleansing applications, the sensation during use after cleansing is emphasized, and further improvement is required for conventional cleansing compositions.

### CITATION LIST

### Patent Literature

Patent Literature 1: JP 2005-120298 A

Patent Literature 2: JP 2000-404590 A

Patent Literature 3: JP 2001-139414 A

### SUMMARY OF THE INVENTION

### Object of the Invention

In view of the above problems, the present invention provides a cleansing agent that can generate foam having excellent texture and can give an excellent sensation during use after cleansing.

### Solution to Problem

According to the present invention, the following invention is provided.

[1] A cleansing agent including:
(A) an anionic surfactant;
(B) a wax having a melting point of 60° C. to 93° C.; and
(C) a cationic polymer.

[2] The cleansing agent according to [1], in which the anion surfactant (A) is an alkali metal salt of a higher fatty acid.

[3] The cleansing agent according to [1] or [2], in which the wax (B) has a melting point of 60° C. to 93° C.

[4] The cleansing agent according to any one of [1] to [3], in which the wax is selected from the group consisting of a microcrystalline wax, a paraffin wax, a polyethylene wax, petrolatum, a synthetic wax, a rice bran wax, a candelilla wax, a yellow beeswax, and a carnauba wax.

[5] The cleansing agent according to any one of [1] to [4], in which the polymer (C) having a cationic group is a hydrophilic polymer having an amino group or a quaternary ammonium group.

[6] The cleansing agent according to any one of [1] to [5], in which
based on a total mass of the cleansing agent,
a content proportion of the anionic surfactant is 15% to 40% by mass,
a content proportion of the wax (B) is 0.3% to 3% by mass, and
a content proportion of the cationic polymer (C) is 0.1% to 1.2% by mass.

[7] The cleansing agent according to any one of [1] to [6], further including (D) an amphoteric surfactant.

[8] The cleansing agent according to [7], in which a content proportion of the amphoteric surfactant (D) is 2% to 4% by mass, based on a total mass of the cleansing agent.

[9] The cleansing agent according to any one of [1] to [8], which is a skin cleansing agent.

[10] The cleansing agent according to any one of [1] to [9], which is a viscous liquid cleansing agent or a paste-like cleansing agent.

### Effects of the Invention

According to the present invention, a cleansing agent excellent in ease of foaming at the time of use and ease of rinsing after use is provided. Furthermore, the foam formed by the cleansing agent according to the present invention has excellent foam elasticity. Furthermore, the cleansing agent according to the present invention can give a high lustrous feeling to the skin after use.

### Mode for Carrying out the Invention

A cleansing agent according to the present invention includes:
as essential components,
(A) an anionic surfactant;
(B) a wax having a melting point of 60° C. to 93° C.; and
(C) a cationic polymer.

### (A) Anionic Surfactant

The anionic surfactant that can be used in the present invention can be arbitrarily selected from those generally used as a cleansing agent material. Specific examples thereof include (i) a carboxylic acid type anionic surfactant having a carboxyl group (—COOH) or a carboxylate group (—COO⁻), (ii) a sulfonic acid type anionic surfactant having a sulfone group (—SO₃H) or a sulfonate (—SO3⁻) group, and (iii) a sulfate type anionic surfactant having a sulfate group (—O—SO₃⁻). Among these, in the present invention, a carboxylic acid type anion surfactant is preferably used, and in the present invention, it is more preferable that the anion surfactant (A) be an alkali metal salt of a higher fatty acid.
(i) The alkali metal salt of a higher fatty acid that can be used in the present invention is a reaction product of a higher fatty acid and an alkali metal compound that are generally used as a cleansing agent material, but a mixture of a higher fatty acid and an alkali metal compound that functions as a neutralizing agent may be used. In addition, what is generally referred to as a higher fatty acid soap is a mixture of a higher fatty acid and an alkali salt thereof, and an alkali metal salt of a higher fatty acid can also be used in the present invention.

The higher fatty acid is preferably a fatty acid having 8 to 22 carbon atoms, and may be any of linear, branched, saturated, and unsaturated fatty acids. Specifically, one or a combination of two or more of behenic acid, palmitoleic acid, oleic acid, isomyristic acid, isopalmitic acid, and isostearic acid can be used.

Examples of an alkali constituting a salt of a higher fatty acid include inorganic alkalis such as potassium hydroxide and sodium hydroxide, organic alkalis such as triethanolamine, and basic amino acids such as lysine and arginine. Among these alkalis, potassium hydroxide is preferable.

As a salt of a higher fatty acid, it is preferable to use potassium stearate, potassium laurate, potassium myristate, or potassium palmitate, and it is also preferable to include all of these four higher fatty acid salts. Furthermore, a mixture of potassium hydroxide with a mixture of stearic acid, lauric acid, myristic acid, and palmitic acid can also be used.

A salt of a higher fatty acid is produced, for example, by neutralizing a higher fatty acid with an alkali, such as an alkali metal compound, but the higher fatty acid is generally not completely neutralized, and a product thereof also contains unreacted higher fatty acids. A neutralization ratio of the higher fatty acid soap that can be used in the present invention is also preferably 60% to 90% by mol. Therefore, in the present invention, it is preferable that the higher fatty acid or a salt (a) thereof contain 60% to 90% by mol of a higher fatty acid alkali salt and 10% to 40% by mol of a higher fatty acid. In particular, it is more preferable that the higher fatty acid or the salt (a) thereof contain about 80% of a higher fatty acid alkali salt. The higher fatty acid or the salt (a) thereof does not need to be a neutralized product of the higher fatty acid, and may be a mixture of a salt of a higher fatty acid and a higher fatty acid at the above ratio.

In addition, as the carboxylic acid type anionic surfactant, a surfactant containing an oxy group (—O—) or an amide group (—NH—C(═O)—) in a hydrocarbon chain can also be used. Examples of such carboxylic acid type anionic surfactants include alkali metal salts such as those of cocoylglycinate, lauroylglutamic acid, myristoylglutamic acid, coconut oil fatty acid acylglutamic acid, coconut oil fatty acid acylglutamic acid, and coconut oil fatty acid acylglycinate, and alkali metal salts of alkoxylated fatty acids such as laureth-6 carboxylic acid, laureth-4 carboxylic acid, and ceteareth-4 carboxylic acid.
(ii) In addition, as the sulfonic acid type anionic surfactant, alkali metal salts such as alkyl sulfates, alkyl ether sulfates, and alkyl amide ether sulfates, which have 6 to 30 carbon atoms, for example, sodium laureth sulfate and the like can be used.
(iii) In addition, as the sulfate type anionic surfactant, coconut fatty acid N-methyltaurine salt lauryl methyltaurine salt, myristyl methyltaurine salt, coconut fatty acid taurine sodium salt, coconut oil fatty acid isethionate, and the like can be used.

Among these anionic surfactants, (i) the alkali metal salt of a higher fatty acid tends to be easily removed by rinsing after cleansing, and is preferable in terms of excellent usability.

In the cleansing agent according to the present invention, a content proportion of the anionic surfactant is preferably 15% to 40% by mass, and more preferably 20% to 30% by mass, based on a total mass of the cleansing agent.

### (B) Wax

In the present invention, the “wax” refers to an oily material that is non-liquid. A melting point of the wax contained in the cleansing agent according to the present invention is 60° C. to 93° C., and preferably 60° C. to 80° C. As the melting point becomes lower, the manufacturability tends to be better. In addition, as the melting point of the wax becomes higher, the foam elasticity and the smooth feeling of the skin after cleansing tend to be improved.

The wax can also be selected and used as necessary from those used in normal cosmetic materials. Specific examples thereof include a microcrystalline wax, a paraffin wax, a polyethylene wax, petrolatum, synthetic wax such as synthetic paraffin, a rice bran wax, a candelilla wax, a yellow beeswax, a carnauba wax, a sunflower seed wax, a cotton wax, a bayberry wax, an insect wax, a spermaceti wax, a montan wax, a kapok wax, a Japan wax, a lanolin acetate, a liquid lanolin, a sugar beet wax, isopropyl lanolin fatty acids, hexyl laurate, reduced lanolin, jojoba wax, hard lanolin, a shellac wax, a beeswax, POE lanolin alcohol ether, POE lanolin alcohol acetate, POE cholesterol ether, lanolin fatty acid polyethylene glycol, fatty acid glyceride, hydrogenated castor oil, petroleum jelly, and POE hydrogenated lanolin alcohol ether. Among these, a wax selected from the group consisting of a microcrystalline wax, a paraffin wax, a polyethylene wax, petrolatum, a synthetic wax, a rice bran wax, a candelilla wax, a yellow beeswax, and a carnauba wax. In the present invention, the wax can be used as a combination of the above-mentioned waxes or other waxes.

In the cleansing agent according to the present invention, a content proportion of the wax is preferably 0.3% to 3% by mass, and more preferably 0.5% to 1% by mass, based on a total mass of the cleansing agent. As a content of the wax becomes higher, the foam elasticity and the smooth feeling of the skin after use tend to be improved, and when the content of the wax is set to be equal to or less than an upper limit, the ease of foaming tends to be improved, and the manufacturability tends to be improved.

### (C) Cationic Polymer

The cationic polymer that can be used in the cleansing agent according to the present invention is not particularly limited, and is preferably a hydrophilic polymer having an amino group or a quaternary ammonium group. Examples of such polymers include a polymer obtained by adding a substituent having an amino group or a quaternary ammonium group to a hydrophilic polymer such as cellulose, and a copolymer containing acrylamide having a cationic group in a side chain, a diallyldialkyl quaternary ammonium salt, or the like as a polymerization unit. Among these, a copolymer containing a diallyldialkyl quaternary ammonium salt as a polymerization unit is preferable, a copolymer containing a diallyldialkyl quaternary ammonium salt and acrylamide as polymerization units is more preferable, and a copolymer containing a diallyldialkyl quaternary ammonium salt, acrylamide, and acrylic acid as polymerization units is still more preferable.

Specific examples thereof include cationized cellulose, cationized starch, a diallyldialkyl quaternary ammonium salt/acrylamide copolymer, and a diallyldialkyl quaternary ammonium salt/acrylamide/acrylic acid copolymer, and a diallyldialkyl quaternary ammonium salt/acrylamide copolymer and a diallyldialkyl quaternary ammonium salt/acrylamide/acrylic acid copolymer are preferable, and a diallyldialkyl quaternary ammonium salt/acrylamide/acrylic acid copolymer is more preferable. In the present invention, the cationic polymer is required to have a cationic group, but the polymer itself is not required to be a cationic polymer. Rather, in the present invention, when an amphoteric polymer such as a diallyldialkyl quaternary ammonium salt/acrylamide/acrylic acid copolymer is used, more excellent effects tend to be obtained.

Examples of the cationic polymer which may be contained in the cleansing agent of the present invention include:
Merquat 100 (trade name: manufactured by Nalco Japan Co., Ltd., cosmetic product display name: Polyquaternium-6),
Merquat 550 (trade name: manufactured by Nalco Japan Co., Ltd., cosmetic product display name: Polyquaternium-7),
Merquat 2200 (trade name: manufactured by Nalco Japan Co., Ltd., cosmetic product display name: Polyquaternium-7), Merquat 3330 (trade name: manufactured by Nalco Japan Co., Ltd., cosmetic product display name: Polyquaternium-39),
trimethylammonio hydroxypropyl hydroxyethyl cellulose chloride (cosmetic display name: Polyquaternium-10), and
guar hydroxypropyltrimonium chloride.

In the cleansing agent according to the present invention, a content proportion of the cationic polymer is preferably 0.1% to 1.2% by mass, and more preferably 0.3% to 1.0% by mass, based on a total mass of the cleansing agent. When a content of the cationic polymer is increased, the foam elasticity tends to be improved, and when the content of the cationic polymer is less than an upper limit, the ease of foaming, the smooth feeling of the skin after use, the ease of rinsing, and the like are improved, and further the manufacturability tends to be improved.

### (D) Amphoteric Surfactant

A cleaning agent according to the present invention can further include an amphoteric surfactant. The amphoteric surfactant that can be used is not particularly limited, and examples thereof include
betaine-based surfactants (for example, lauryl betaine, coco betaine, imidazolium betaine, cocamidopropyl betaine, 2-heptadecyl-N-carboxymethyl-N-hydroxyethylimidazolinium betaine, lauryldimethylaminoacetic acid betaine, alkyl betaine, amide betaine, and sulfobetaine); and
imidazoline-based amphoteric surfactants (for example, 2-undecyl-N,N,N-(hydroxyethylcarboxymethyl)-2-imidazoline sodium and 2-cocoyl-2-imidazolinium hydroxide-1-carboxyethyloxy disodium salt),
preferably include betaine-based surfactants, and more preferably include lauryl betaine, coco betaine, imidazolium betaine, and cocamidopropyl betaine.

In the cleansing agent according to the present invention, the amphoteric surfactant has an effect of remarkably improving the smooth feeling of the skin after use and the ease of foaming. In addition, an effect of improving the high-temperature stability of the cleansing agent is also provided. In a case where the amphoteric surfactant is used, a content proportion thereof is preferably 2% to 4% by mass based on a total mass of the cleansing agent. When increasing a content of the amphoteric surfactant, the effects can be more strongly exhibited. On the other hand, the content of the amphoteric surfactant is preferably set to be equal to or less than an upper limit, in order to sufficiently exhibit the effects of improving the foam elasticity and the smooth feeling of the skin after use.

### (E) Water

The cleansing agent according to the present invention generally further includes water in addition to the above components. As the water, water used for cosmetics, quasi-drugs, and the like can be used, and for example, purified water, ion-exchanged water, tap water, and the like can be used.

### (F) Other Additives

The cleansing agent according to the present invention can include various additives as necessary, in addition to the above components. Examples of such additives include the following additives, and the additives can be produced by further incorporating one or two or more kinds thereof, within a range where the effect of the present invention is not impaired.

The cleansing agent according to the present invention can include a nonionic surfactant. Examples of the nonionic surfactant that can be used include fatty acid alkanolamides such as coconut oil fatty acid monoethanolamide, coconut oil fatty acid diethanolamide, lauric acid isopropanolamide, oleic acid diethanolamide; sorbitan fatty acid esters such as sorbitan monostearate and sorbitan sesquioleate; alkylene glycol fatty acid esters such as diethylene glycol laurate, propylene glycol laurate, ethylene glycol monooleate, and ethylene glycol distearate; POE sorbitan fatty acid esters such as hydrogenated castor oil derivatives, glycerin alkyl ethers, POE sorbitan monooleate, polyoxyethylene sorbitan monostearate; POE-sorbitol fatty acid esters such as POE-sorbitol monolaurate; POE glycerin fatty acid esters such as POE-glycerin monoisostearate; POE glycerin fatty acid esters such as polyethylene glycol monooleate and POE distearate; POE alkyl ethers such as POE-octyldodecyl ether; POE alkylphenyl ethers such as POE nonylphenyl ether; POE/POP alkyl ethers; pluronic type; POE castor oil; POE hydrogenated castor oil derivative; sugars such as sugar esters, sugar ethers, and sugar amides; and alkyl glycoside, among these, one or two or more kinds thereof may be combined and incorporated, in particular, alkylene glycol fatty acid esters are preferable, and diethylene glycol laurate is still more preferable.

In a case where the cleansing agent according to the present invention includes a nonionic surfactant, a content proportion thereof is preferably 0.1% to 15% by mass, and more preferably 0.3% to 10% by mass, based on a total mass of the cleansing agent.

The cleansing agent according to the present invention can include a moisturizing agent. Examples of the moisturizing agent include polyethylene glycol, propylene glycol, dipropylene glycol, glycerin, 1,3-butylene glycol, xylitol, sorbitol, maltitol, chondroitin sulfate, hyaluronic acid, mucoitin sulfate, charonic acid, atelocollagen, cholesteryl-12-hydroxystearate, sodium lactate, bile salts, dl-pyrrolidone carboxylate, short-chain soluble collagen, a diglycerin(EO)PO adduct, a chestnut rose extract, a yarrow extract, and a melilot extract.

The cleansing agent according to the present invention can include a powder component. Examples of the powder component include an inorganic powder (for example, talc, kaolin, mica, sericite (sericite), muscovite, phlogopite, synthetic mica, red mica, biotite, vermiculite, magnesium carbonate, calcium carbonate, aluminum silicate, barium silicate, calcium silicate, magnesium silicate, strontium silicate, metal tungstate, magnesium, silica, zeolite, barium sulfate, calcined calcium sulfate (calcined gypsum), calcium phosphate, fluorapatite, hydroxyapatite, ceramic powders, metal soaps (for example, zinc myristate, calcium palmitate, and aluminum stearate), boron nitride, and the like); an organic powder (for example, a polyamide resin powder (a nylon powder), a polyethylene powder, a polymethyl methacrylate powder, a polystyrene powder, a copolymer resin powder of styrene and acrylic acid, a benzoguanamine resin powder, a polytetrafluoroethylene powder, a cellulose powder, and the like); an inorganic white-based pigment (for example, titanium dioxide, zinc oxide, and the like); an inorganic red-based pigment (for example, iron oxide (Bengala), iron titanate, and the like); an inorganic brown-based pigment (for example, y-iron oxide and the like); an inorganic yellow-based pigment (for example, yellow iron oxide, yellow earth, and the like); an inorganic black-based pigment (for example, black iron oxide, carbon, lower titanium oxide, and the like); an inorganic violet-based pigment (for example, mango violet, cobalt violet, and the like); an inorganic green-based pigment (for example, chromium oxide, chromium hydroxide, cobalt titanate, and the like); an inorganic blue-based pigment (for example, ultramarine blue, Prussian blue, and the like); a pearl pigment (for example, titanium oxide-coated mica, titanium oxide-coated bismuth oxychloride, titanium oxide-coated talc, colored titanium oxide-coated mica, bismuth oxychloride, fish scale foil, and the like); a metallic powder pigment (for example, an aluminum powder, a kappa powder, and the like); an organic pigment such as zirconium, barium, or aluminum lake (for example, an organic pigment such as Red No. 201, Red No. 202, Red No. 204, Red No. 205, Red No. 220, Red No. 226, Red No. 228, Red No. 405, Orange No. 203, Orange No. 204, Yellow No. 205, Yellow No. 401, and Blue No. 404, Red No. 3, Red No. 104, Red No. 106, Red No. 227, Red No. 230, Red No. 401, Red No. 505, Orange No. 205, Yellow No. 4, Yellow No. 5, Yellow No. 202, Yellow No. 203, Green No. 3, and Blue No. 1, and the like); natural color (for example, chlorophyll, β-carotene, and the like); and the like.

The cleansing agent according to the present invention can include an oil component. Examples of the oil component include liquid oil-and-fat, solid oil-and-fat, hydrocarbon oil, synthetic ester oil, and silicone oil. The wax (B) is excluded from the oil component in the present invention.

Examples of the liquid oil-and-fat include avocado oil, camellia oil, turtle oil, macadamia nut oil, corn oil, mink oil, olive oil, rapeseed oil, egg yolk oil, sesame oil, persic oil, wheat germ oil, sasanqua oil, castor oil, linseed oil, safflower oil, cottonseed oil, eno oil, soybean oil, peanut oil, tea seed oil, kaya oil, rice bran oil, cinnamon oil, Japanese tung oil, jojoba oil, germ oil, triglycerin, and the like.

Examples of the solid oil-and-fat include cocoa butter, coconut oil, horse fat, hydrogenated coconut oil, palm oil, beef tallow, mutton fat, hardened beef tallow, palm kernel oil, lard, beef bone fat, wintergreen kernel oil, hydrogenated oil, beef leg fat, Japan wax, and hardened castor oil.

Examples of the hydrocarbon oil include liquid paraffin, ozokerite, squalane, pristane, paraffin, ceresin, squalene, petrolatum, and microcrystalline wax.

Examples of synthetic ester oil include isopropyl myristate, cetyl octoate, octyldodecyl myristate, isopropyl palmitate, butyl stearate, hexyl laurate, myristyl myristate, decyl oleate, hexyldecyl dimethyloctanoate, cetyl lactate, myristyl lactate, lanolin acetate, isocetyl stearate, isocetyl isostearate, cholesteryl 12-hydroxystearate, ethylene glycol di-2-ethylhexanoate, dipentaerythritol fatty acid ester, N-alkyl glycol monoisostearate, neopentyl glycol dicaprate, diisostearyl malate, glycerin di-2-heptylundecanoate, trimethylolpropane tri-2-ethylhexanoate, trimethylolpropane triisostearate, pentaerythritol tetra-2-ethylhexanoate, glycerin tri-2-ethylhexanoate, glyceryl trioctanoate, glycerin triisopalmitate, trimethylolpropane triisostearate, cetyl 2-ethylhexanoate, 2-ethylhexyl palmitate, glyceryl trimyristate, tri-2-heptylundecanoic acid glyceride, castor oil fatty acid methyl ester, oleyl oleate, acetoglyceride, 2-heptylundecyl palmitate, diisobutyl adipate, N-lauroyl-L-glutamic acid-2-octyldodecyl ester, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, and triethyl citrate.

Examples of the silicone oil include chain polysiloxane (for example, dimethylpolysiloxane, methylphenylpolysiloxane, diphenylpolysiloxane, and the like), cyclic polysiloxane (for example, octamethylcyclotetrasiloxane, decamethylcyclopentasiloxane, dodecamethylcyclohexasiloxane, and the like), silicone resin forming a three-dimensional network structure, silicone rubber, and various modified polysiloxanes (amino-modified polysiloxane, polyether-modified polysiloxane, alkyl-modified polysiloxane, fluorine-modified polysiloxane, and the like).

The cleansing agent according to the present invention can include a water-soluble polymer. Such water-soluble polymers include natural, semi-synthetic, or synthetic water-soluble polymers.

Examples of the natural water-soluble polymer include plant-based polymers (for example, gum arabic, gum tragacanth, galactan, guar gum, carob gum, karaya gum, carrageenan, pectin, agar, quince seed (marmelo), algae colloid (brown algae extract), starch (rice, corn, potato, wheat), glycyrrhizic acid); microbial-based polymers (for example, xanthan gum, dextran, succinoglucan, pullulan, and the like); and animal-based polymers (for example, collagen, casein, albumin, gelatin, and the like).

Examples of the semi-synthetic water-soluble polymer include starch-based polymers (for example, carboxymethyl starch, methyl hydroxypropyl starch, and the like); cellulose-based polymers (methyl cellulose, ethyl cellulose, methyl hydroxypropyl cellulose, hydroxyethyl cellulose, sodium cellulose sulfate, hydroxypropyl cellulose, carboxymethyl cellulose, sodium carboxymethyl cellulose, crystalline cellulose, cellulose powder, and the like); and an alginic acid-based polymers (for example, sodium alginate, propylene glycol alginate ester, and the like).

Examples of the synthetic water-soluble polymer include vinyl-based polymers (for example, polyvinyl alcohol, polyvinyl methyl ether, polyvinyl pyrrolidone, carboxyvinyl polymer, and the like); polyoxyethylene-based polymers (for example, polyoxyethylene polyoxypropylene copolymers of polyethylene glycol 20,000, 40,000, and 60,000, and the like); acrylic polymer (for example, sodium polyacrylate, polyethyl acrylate, polyacrylamide, and the like); polyethyleneimine; and cationic polymers.

The cleansing agent according to the present invention can include a thickener. Examples of the thickener include gum arabic, carrageenan, karaya gum, tragacanth gum, carob gum, quince seed (marmelo), casein, dextrin, gelatin, sodium pectinate, sodium araginate, methyl cellulose, ethyl cellulose, CMC, hydroxyethyl cellulose, hydroxypropyl cellulose, PVA, PVM, PVP, a carboxyvinyl polymer, locust bean gum, guar gum, tamarind gum, dialkyldimethylammonium sulfate cellulose, xanthan gum, magnesium aluminum silicate, bentonite, hectorite, A1 Mg silicate (VEEGUM), laponite, and silicic anhydride.

The cleansing agent according to the present invention can include an ultraviolet absorber. Examples of the ultraviolet absorber include benzoic acid-based ultraviolet absorbers (for example, para-aminobenzoic acid (hereinafter, abbreviated as PABA), PABA monoglycerin ester, N,N-dipropoxy PABA ethyl ester, N,N-diethoxy PABA ethyl ester, N,N-dimethyl PABA ethyl ester, N,N-dimethyl PABA butyl ester, N,N-dimethyl PABA ethyl ester, and the like); anthranilic acid-based ultraviolet absorbers (for example, homomenthyl-N-acetylanthranilate, and the like); salicylic acid-based ultraviolet absorbers (for example, amyl salicylate, menthyl salicylate, homomenthyl salicylate, octyl salicylate, phenyl salicylate, benzyl salicylate, p-isopropanol phenyl salicylate, and the like); cinnamic acid ultraviolet absorbers (for example, octyl cinnamate, ethyl-4-isopropyl cinnamate, methyl-2,5-diisopropyl cinnamate, ethyl-2,4-diisopropyl cinnamate, methyl-2,4-diisopropyl cinnamate, propyl-p-methoxycinnamate, isopropyl-p-methoxycinnamate, isoamyl-p-methoxycinnamate, octyl-p-methoxycinnamate (2-ethylhexyl-p-methoxycinnamate), 2-ethoxyethyl-p-methoxycinnamate, cyclohexyl-p-methoxycinnamate, ethyl-α-cyano-β-phenylcinnamate, 2-ethylhexyl-α-cyano-β-phenylcinnamate, glycerylmono-2-ethylhexanoyl-dipara methoxycinnamate, and the like); benzophenone-based ultraviolet absorbers (for example, 2,4-dihydroxybenzophenone, 2,2′-dihydroxy-4-methoxybenzophenone, 2,2′-dihydroxy-4,4′-dimethoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone-5-sulfonate, 4-phenylbenzophenone, 2-ethylhexyl-4′-phenyl-benzophenone-2-carboxylate, 2-hydroxy-4-n-octoxybenzophenone, 4-hydroxy-3-carboxybenzophenone, and the like); 3-(4′-methylbenzylidene)-d, I-camphor, 3-benzylidene-d, I-camphor; 2-phenyl-5-methylbenzoxazole;2,2′-hydroxy-5-methylphenylbenzotriazole; 2-(2′-hydroxy-5′-t-octylphenyl) benzotriazole; 2-(2′-hydroxy-5′-methylphenylbenzotriazole; dibenzalazine; dianisoylmethane; 4-methoxy-4′-t-butyldibenzoylmethane; and 5-(3,3-dimethyl-2-norbornylidene)-3-pentan-2-one.

The cleansing agent according to the present invention can include a sequestering agent. Examples of the sequestering agent include 1-hydroxyethane-1,1-diphosphonic acid, 1-hydroxyethane-1,1-diphosphonic acid tetrasodium salt, edetate disodium, edetate trisodium, edetate tetrasodium, sodium citrate, sodium polyphosphate, sodium metaphosphate, gluconic acid, phosphoric acid, citric acid, ascorbic acid, succinic acid, edetic acid, ethylenediamine hydroxyethyl triacetate trisodium, and ethylenediamine tetraacetate disodium.

The cleansing agent according to the present invention can include an alcohol. The alcohol may be either a lower alcohol or a higher alcohol, and may be either a monohydric alcohol or a polyhydric alcohol.

Examples of the lower alcohol include ethanol, propanol, isopropanol, isobutyl alcohol, and t-butyl alcohol.

Examples of the higher alcohol include linear alcohols (for example, lauryl alcohol, cetyl alcohol, stearyl alcohol, behenyl alcohol, myristyl alcohol, oleyl alcohol, cetostearyl alcohol, and the like); and branched chain alcohols (for example, monostearyl glycerin ether (butyl alcohol), 2-decyltetradecinol, lanolin alcohol, cholesterol, phytosterol, hexyldodecanol, isostearyl alcohol, octyldodecanol, and the like).

Examples of the polyhydric alcohol include dihydric alcohols (for example, ethylene glycol, propylene glycol, trimethylene glycol, 1,2-butylene glycol, 1,3-butylene glycol, tetramethylene glycol, 2,3-butylene glycol, pentamethylene glycol, 2-butene-1,4-diol, hexylene glycol, octylene glycol, and the like); trihydric alcohols (for example, glycerin, trimethylolpropane, and the like); tetrahydric alcohols (for example, pentaerythritol, such as 1,2,6-hexanetriol, and the like); pentahydric alcohol (for example,xylitol, and the like); hexahydric alcohols (for example, sorbitol, mannitol, and the like); polyhydric alcohol polymers (for example, diethylene glycol, dipropylene glycol, triethylene glycol, polypropylene glycol, tetraethylene glycol, diglycerin, polyethylene glycol, triglycerin, tetraglycerin, polyglycerin, and the like); divalent alcohol alkyl ethers (for example, ethylene glycol monomethyl ether, ethylene glycol monoethyl ether, ethylene glycol monobutyl ether, ethylene glycol monophenyl ether, ethylene glycol monohexyl ether, ethylene glycol mono 2-methylhexyl ether, ethylene glycol isoamyl ether, ethylene glycol benzyl ether, ethylene glycol isopropyl ether, ethylene glycol dimethyl ether, ethylene glycol diethyl ether, ethylene glycol dibutyl ether, and the like); dihydric alcohol alkyl ethers (for example, diethylene glycol monomethyl ether, diethylene glycol monoethyl ether, diethylene glycol monobutyl ether, diethylene glycol dimethyl ether, diethylene glycol diethyl ether, diethylene glycol butyl ether, diethylene glycol methyl ethyl ether, triethylene glycol monomethyl ether, triethylene glycol monoethyl ether, propylene glycol monomethyl ether, propylene glycol monoethyl ether, propylene glycol monobutyl ether, propylene glycol isopropyl ether, dipropylene glycol methyl ether, dipropylene glycol ethyl ether, dipropylene glycol butyl ether, and the like); dihydric alcohol ether esters (for example, ethylene glycol monomethyl ether acetate, ethylene glycol monoethyl ether acetate, ethylene glycol monobutyl ether acetate, ethylene glycol monophenyl ether acetate, ethylene glycol diadipate, ethylene glycol disuccinate, diethylene glycol monoethyl ether acetate, diethylene glycol monobutyl ether acetate, propylene glycol monomethyl ether acetate, propylene glycol monoethyl ether acetate, propylene glycol monopropyl ether acetate, propylene glycol monophenyl ether acetate, and the like); glycerin monoalkyl ether (for example, xyl alcohol, selachyl alcohol, batyl alcohol, and the like); sugar alcohols (for example, sorbitol, maltitol, maltotriose, mannitol, sucrose, erythritol, glucose, fructose, starch sugars, maltose, xylitose, starch sugar reducing alcohols, and the like); glysolid; tetrahydrofurfuryl alcohol; POE-tetrahydrofurfuryl alcohol; POP-butyl ether; POP/POE-butyl ether; tripolyoxypropylene glycerin ether; POP-glycerin ether; POP-glycerin ether phosphate; and POP/POE-pentane erythritol ether, polyglycerin, and the like. The polyhydric alcohol may function as a moisturizing agent.

The cleansing agent according to the present invention can include a saccharide selected from monosaccharides or polysaccharides.

Examples of the monosaccharide include trioses (for example, D-glycerylaldehyde, dihydroxyacetone, and the like); tetroses (for example, D-erythrose, D-erythrulose, D-threose, erythritol, and the like); pentoses (for example, L-arabinose, D-xylose, L-lyxose, D-arabinose, D-ribose, D-ribulose, D-xylulose, L-xylulose, and the like); hexoses (for example, D-glucose, D-talose, D-busicose, D-galactose, D-fructose, L-galactose, L-mannose, D-tagatose, and the like); heptoses (for example, aldoheptose, heprose, and the like); octoses (for example, octulose or the like); deoxy sugars (for example, 2-deoxy-D-ribose, 6-deoxy-L-galactose, 6-deoxy-L-mannose, and the like); amino sugars (for example, D-glucosamine, D-galactosamine, sialic acid, aminouronic acid, muramic acid, and the like); and uronic acids (for example, D-glucuronic acid, D-mannuronic acid, L-guluronic acid, D-galacturonic acid, L-iduronic acid, and the like).

Examples of the oligosaccharide include sucrose, gentianose, umbelliferose, lactose, planteose, isolychnoses, α,α-trehalose, raffinose, lychnoses, umbilicin, stachyose, and verbascose.

Examples of the polysaccharide include cellulose, quince seed, chondroitin sulfate, starch, galactan, dermatan sulfate, glycogen, gum arabic, heparan sulfate, hyaluronic acid, tragacanth gum, keratan sulfate, chondroitin, xanthan gum, mucoitin sulfate, guar gum, dextran, keratosulfate, locust bean gum, succinoglucan, and caronic acid.

The cleansing agent according to the present invention can include an amino acid. Examples of the amino acid include neutral amino acids (for example, threonine, cysteine, and the like); and basic amino acids (for example, hydroxylysine and the like). Also, examples of an amino acid derivative include sodium acyl sarcosine (sodium lauroyl sarcosine), acyl glutamate, sodium acyl β-alanine, glutathione, and pyrrolidone carboxylic acid.

The cleansing agent according to the present invention can include an organic amine. Examples of the organic amine include monoethanolamine, diethanolamine, triethanolamine, morpholine, triisopropanolamine, 2-amino-2-methyl-1,3-propanediol, and 2-amino-2-methyl-1-propanol.

The cleansing agent according to the present invention can include a polymeric emulsion. Examples of the polymer emulsion include an acrylic resin emulsion, a polyacrylic acid ethyl emulsion, an acrylic resin liquid, a polyacrylic alkyl ester emulsion, a polyvinyl acetate resin emulsion, and a natural rubber latex.

The cleansing agent according to the present invention can include a pH adjusting agent. As the pH adjusting agent, an acid or an alkali can be used, but a buffer material can also be used. Examples thereof include buffer agents such as lactic acid-sodium lactate, citric acid-sodium citrate, succinic acid-sodium succinate. Examples of vitamins include vitamins A, B1, B2, B6, C, and E and derivatives thereof, pantothenic acid and derivatives thereof, and biotin.

The cleansing agent according to the present invention can include an antioxidant. Examples of the antioxidant include tocopherols, dibutylhydroxytoluene, butylhydroxyanisole, gallic acid esters, sulfites, and bisulfites. Examples of an antioxidant auxiliary include phosphoric acid, citric acid, ascorbic acid, maleic acid, malonic acid, succinic acid, fumaric acid, kephalin, hexametaphosphate, phytic acid, and ethylenediaminetetraacetic acid.

Examples of other components that can be incorporated include preservatives (ethylparaben, butylparaben, and the like); anti-inflammatory agents (for example, a glycyrrhizic acid derivative, a glycyrrhetinic acid derivative, a salicylic acid derivative, hinokitiol, zinc oxide, allantoin, and the like); whitening agents (for example, a placenta extract, a saxifrage extract, arbutin, and the like); various extracts (for example, amur cork tree, coptis japonica, gromwell, peony, swertia japonica, birch, sage, loquat, carrots, aloe, mallow, iris, grapes, coix seed, loofah, lily, saffron, Cnidium officinale, ginger, hypericum, Ononis, garlic, red pepper, citrus unshiu peel, Japanese angelica, seaweed, and the like), activators (for example, royal jelly, photosensitizer, a cholesterol derivative, and the like); blood circulation promoters (for example, nonylic acid valenylamide, nicotinic acid benzyl ester, nicotinic acid β-butoxyethyl ester, capsaicin, zingerone, cantharides tincture, ichthammol, tannic acid, α-borneol, tocopherol nicotinate, inositol hexanicotinate, cyclandelate, cinnarizine, tolazoline, acetylcholine, verapamil, cepharanthine, γ-oryzanol, and the like); antiseborrheic agents (for example, sulfur, thianthol, and the like); anti-inflammatory agents (for example, tranexamic acid, thiotaurine, hypotaurine, and the like).

According to a preferred aspect of the cleansing agent according to the present invention, a viscous liquid cleansing agent or a paste-like cleansing agent is provided. These cleansing agents are in a liquid to semi-solid form having fluidity, and preferably have a viscosity (B-type viscometer, 30° C.) of 1,000 mPa·s or more.

The cleansing agent according to the present invention is preferably a skin cleansing agent. As an embodiment, it is suitably used for cleansing the skin after makeup application after going out, and a dosage form thereof preferably takes a lotion form, a gel form, or a cream form, and more preferably takes a cream form in particular.

According to a preferred aspect of the cleansing agent according to the present invention, a makeup removing cleansing agent is provided. Makeup includes a sunblock such as a sunscreen in addition to lipstick, foundation, and the like.

According to a preferred embodiment of the cleansing agent according to the present invention, a sebum cleansing agent is provided. Sebum is derived from sebaceous glands or horny layer cells and protects skin, but since when sebum is left as it is for a long time, the sebum may be oxidized by ultraviolet rays or the like and adversely affect the skin, it is preferable to remove such sebum stains with the cleansing agent according to the present invention.

The dosage form of the cleansing agent according to the present invention is arbitrary, and may be any dosage form such as a solution system, a solubilizing system, an emulsifying system, a powder dispersion system, a water 2-oil bilayer system, or a water-oil-powder trilayer system.

### EXAMPLES

The present invention will be specifically described based on the following examples, but the present invention is not limited to these examples. The content proportion is represented by % by mass unless otherwise specified.

### Examples 1 to 24 and Comparative Examples 1 to 3

The cleansing agents of Examples 1 to 24 and Comparative Examples 1 to 3 were prepared with the incorporations shown in each table.

Furthermore, the performance of these cleansing agents was evaluated according to the following criteria.

### [Ease of Foaming]

1 g of each cleansing agent was mixed with 20 g of water and foaming was performed for 1 minute, and then the ease of foaming was evaluated.

S: Foaming is very fast

A: Foaming is fast

B: It is not considered that foaming is slow

C: Foaming is slow

### [Foam Elasticity]

1 g of each cleansing agent was mixed with 20 g of water and foamed, and the elasticity when the formed foam was pressed with a finger was evaluated.

S: Elasticity is felt very much

A: Elasticity is felt

B: Certain degree of elasticity is felt

C: Elasticity is not felt

### [Ease of Rinsing]

1 g of each cleansing agent was mixed with 20 g of water, and after performing foaming for 1 minute, the skin was cleaned with the formed foam, and the ease of rinsing at the time of cleansing was evaluated.

A: Rinsing is easy

B: Rinsing is easy, but slimy or the like is felt

C: Rinsing is difficult, and slimy

### [Smooth Feeling of Skin After Use]

1 g of each cleansing agent was mixed with 20 g of water, and after performing foaming for 1 minute, the feel of the skin after cleansing the skin with the formed foam was evaluated.

S: Skin is very smooth

A: Skin is smooth

B: It is felt when it is told that skin is smooth

C: Smooth feeling of skin is not felt

TABLE 1

| | | Comparative Example | | | | Example | | | | | | | | | | |
|---|---|---|---|---|---|---|---|---|---|---|---|---|---|---|---|---|
| [symbol] | | 1 | 2 | 3 | 4 | 1 | 2 | 3 | 4 | 5 | 6 | 7 | 8 | 9 | 10 | 11 |
| A | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | [symbol] | — | — | — | — | — | — | — | — | — | — | — | — | — | — | — |
| | [symbol] | — | — | — | — | — | — | — | — | — | — | — | — | — | — | — |
| B | [symbol] | — | 1 | — | — | — | — | — | — | — | — | — | — | — | — | — |
| [symbol] | [symbol] | — | — | 1 | — | — | — | — | — | — | — | — | — | — | — | — |
| | [symbol] | — | — | — | 1 | — | — | — | — | — | — | — | — | — | — | — |
| | [symbol] | — | — | — | — | — | — | — | — | — | — | — | — | — | 1 | — |
| | [symbol] | — | — | — | — | 1 | — | — | — | — | — | — | — | — | — | — |
| | [symbol] | — | — | — | — | — | — | — | — | — | — | — | — | 1 | — | — |
| | [symbol] | — | — | — | — | — | 1 | — | — | — | — | — | — | — | — | — |
| | [symbol] | — | — | — | — | — | — | — | — | — | — | — | 1 | — | — | — |
| | [symbol] | — | — | — | — | — | — | [symbol] | [symbol] | [symbol] | [symbol] | [symbol] | — | — | — | — |
| | [symbol] | — | — | — | — | — | — | — | — | — | — | — | — | — | — | [symbol] |
| C | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | — | — | — | — | — | — | — | — | — | — | — | — | — | — | — |
| [symbol] | [symbol] | — | — | — | — | — | — | — | — | — | — | — | — | — | — | — |
| | [symbol] | — | — | — | — | — | — | — | — | — | — | — | — | — | — | — |
| D | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | — | — | — | — | — | — | — | — | — | — | — | — | — | — | — |
| [symbol] | [symbol] | — | — | — | — | — | — | — | — | — | — | — | — | — | — | — |
| | [symbol] | — | — | — | — | — | — | — | — | — | — | — | — | — | — | — |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | A | A | A | A | [symbol] | [symbol] | [symbol] | [symbol] | [symbol] | A | B | [symbol] | [symbol] | A | A |
| | [symbol] | B | [symbol] | C | [symbol] | [symbol] | [symbol] | [symbol] | A | [symbol] | [symbol] | [symbol] | A | A | A | [symbol] |
| | [symbol] | C | [symbol] | C | [symbol] | A | A | [symbol] | A | A | [symbol] | [symbol] | A | A | A | [symbol] |
| | [symbol] | [symbol] | A | A | A | A | A | A | A | A | A | A | A | A | A | A |

[symbol]

indicates data missing or illegible when filed

From the results shown in Table 1, by using a wax having a specific melting point, it is possible to obtain an excellent effect in foam elasticity and smooth feeling of the skin after use. All the cleansing agents were excellent in manufacturability, but Example 7 having a relatively high wax content proportion and Example 11 using a wax having a relatively high melting point tended to be slightly inferior in manufacturability.

TABLE 2

| | | Example | | | | | | | | | | | | | | |
|---|---|---|---|---|---|---|---|---|---|---|---|---|---|---|---|---|
| [symbol] | | 12 | 13 | 14 | 15 | [symbol] | 16 | 17 | 18 | 19 | 20 | [symbol] | 21 | 22 | 23 | 24 |
| A | [symbol] | — | — | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | — | — | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | — | — | [symbol] | [symbol] | [symbol] | [symbol] | [symbol] | [symbol] | [symbol] | [symbol] | [symbol] | [symbol] | [symbol] | [symbol] | [symbol] |
| | [symbol] | — | — | [symbol] | [symbol] | [symbol] | [symbol] | [symbol] | [symbol] | [symbol] | [symbol] | [symbol] | [symbol] | [symbol] | [symbol] | [symbol] |
| | [symbol] | — | — | [symbol] | [symbol] | [symbol] | [symbol] | [symbol] | [symbol] | [symbol] | [symbol] | [symbol] | [symbol] | [symbol] | [symbol] | [symbol] |
| | [symbol] | [symbol] | — | — | — | — | — | — | — | — | — | — | — | — | — | — |
| | [symbol] | — | [symbol] | — | — | — | — | — | — | — | — | — | — | — | — | — |
| B | [symbol] | — | — | — | — | — | — | — | — | — | — | — | — | — | — | — |
| [symbol] | [symbol] | — | — | — | — | — | — | — | — | — | — | — | — | — | — | — |
| | [symbol] | — | — | — | — | — | — | — | — | — | — | — | — | — | — | — |
| | [symbol] | — | — | — | — | — | — | — | — | — | — | — | — | — | — | — |
| | [symbol] | — | — | — | — | — | — | — | — | — | — | — | — | — | — | — |
| | [symbol] | — | — | — | — | — | — | — | — | — | — | — | — | — | — | — |
| | [symbol] | — | — | — | — | — | — | — | — | — | — | — | — | — | — | — |
| | [symbol] | — | — | — | — | — | — | — | — | — | — | — | — | — | — | — |
| | [symbol] | [symbol] | [symbol] | 1 | 1 | 1 | 1 | 1 | 1 | 1 | 1 | 1 | 1 | 1 | 1 | 1 |
| | [symbol] | — | — | — | — | — | — | — | — | — | — | — | — | — | — | — |
| C | [symbol] | [symbol] | [symbol] | [symbol] | [symbol] | [symbol] | [symbol] | — | — | — | [symbol] | [symbol] | [symbol] | [symbol] | [symbol] | [symbol] |
| [symbol] | [symbol] | — | — | — | — | — | — | 1 | — | — | — | — | — | — | — | — |
| [symbol] | [symbol] | — | — | — | — | — | — | — | [symbol] | — | — | — | — | — | — | — |
| | [symbol] | — | — | — | — | — | — | — | — | [symbol] | — | — | — | — | — | — |
| D | [symbol] | [symbol] | [symbol] | [symbol] | [symbol] | [symbol] | [symbol] | [symbol] | [symbol] | [symbol] | [symbol] | [symbol] | [symbol] | — | — | — |
| [symbol] | [symbol] | — | — | — | — | — | — | — | — | — | — | — | — | [symbol] | — | — |
| [symbol] | [symbol] | — | — | — | — | — | — | — | — | — | — | — | — | — | [symbol] | — |
| | [symbol] | — | — | — | — | — | — | — | — | — | — | — | — | — | — | [symbol] |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | A | A | [symbol] | A | A | A | A | A | [symbol] | [symbol] | [symbol] | A | A |
| | [symbol] | A | A | A | A | [symbol] | A | [symbol] | [symbol] | [symbol] | A | [symbol] | [symbol] | A | [symbol] | [symbol] |
| | [symbol] | [symbol] | [symbol] | A | A | A | [symbol] | [symbol] | [symbol] | [symbol] | A | [symbol] | [symbol] | A | [symbol] | [symbol] |
| | [symbol] | A | A | A | A | A | [symbol] | A | A | A | A | [symbol] | A | A | A | A |

[symbol]

indicates data missing or illegible when filed

In Table 2, Example 5 in Table 1 is also reproduced for ease of comparison. From the results shown in Table 2, it is found that the effects of the present invention can be obtained even when the kind and content proportion of the anionic surfactant and the cationic polymer are changed. From comparison of Examples 20, 5, and 21, it can be confirmed that when the content proportion of the amphoteric surfactant is increased, the smooth feeling of the skin after use tends to be improved. In addition, it can be confirmed that when the content proportion of the cationic polymer is increased, the smooth feeling of the skin after use and the ease of rinsing tend to be improved. All the cleansing agents were excellent in manufacturability, but Example 8 using Example 19 having a relatively high cationic polymer content proportion tended to be slightly inferior in manufacturability.

1. A cleansing agent comprising:
(A) an anionic surfactant;
(B) a wax having a melting point of 60° C. to 93° C.; and
(C) a cationic polymer.

2. The cleansing agent according to claim 1, wherein the anion surfactant (A) is an alkali metal salt of a higher fatty acid.

3. The cleansing agent according to claim 1 or 2, wherein the wax (B) has a melting point of 60° C. to 93° C.

4. The cleansing agent according to claim 1 or 2, wherein the wax is selected from the group consisting of a microcrystalline wax, a paraffin wax, a polyethylene wax, petrolatum, a synthetic wax, a rice bran wax, a candelilla wax, and a yellow beeswax and a carnauba wax.

5. The cleansing agent according to claim 1 or 2, wherein the polymer (C) having a cationic group is a hydrophilic polymer having an amino group or a quaternary ammonium group.

6. The cleansing agent according to claim 1 or 2, wherein
based on a total mass of the cleansing agent,
a content proportion of the anionic surfactant is 15% to 40% by mass,
a content proportion of the wax (B) is 0.3% to 3% by mass, and
a content proportion of the cationic polymer (C) is 0.1% to 1.2% by mass.

7. The cleansing agent according to claim 1 or 2, further comprising (D) an amphoteric surfactant.

8. The cleansing agent according to claim 7, wherein a content proportion of the amphoteric surfactant (D) is 2% to 4% by mass, based on a total mass of the cleansing agent.

9. The cleansing agent according to claim 1 or 2, which is a skin cleansing agent.

10. The cleansing agent according to claim 1 or 2, which is a viscous liquid cleansing agent or a paste-like cleansing agent.
